(19) United States
(12) Patent Application Publication
(10) Pub. No.: US 2014/0370086 A1
(43) Pub. Date: Dec. 18, 2014

(54) SUBLINGUAL IMMUNOTHERAPY WITH REDUCED ORAL ITCHINESS

(71) Applicants: Ziv Harish, Tenafly, NJ (US); Russ Weinzimmer, Milford, NH (US)

(72) Inventors: Ziv Harish, Tenafly, NJ (US); Russ Weinzimmer, Milford, NH (US)

(21) Appl. No.: 13/919,948

(22) Filed: Jun. 17, 2013

(51) Int. Cl. A61K 47/22; A61K 39/36; A61K 39/35

(52) U.S. Cl. CPC A61K 47/22; A61K 39/35; A61K 39/36
USPC 424/472; 424/275.1; 424/475; 424/400

(57) ABSTRACT

Sublingual immunotherapy (SLIT) with reduced oral itchiness is disclosed. The improved sublingual immunotherapy combines a monotonically increasing dose of allergen, along with a constant dose of mast cell stabilizer, thereby substantially avoiding the oral itchiness and other uncomfortable adverse reactions typically experienced with SLIT, which can improve patient compliance. An antihistamine and/or a leukotriene inhibitor can also be added along with the mast cell stabilizer. Multi-layer and/or coated tablets, and flexible paired ampoules with special features to advantageously time the dose of the allergen relative to the dose of the mast cell stabilizer, have been provided to effectively administer the improved sublingual immunotherapy in a highly convenient manner. Methods, preparations, and apparatuses for administration of SLIT, apparatus for sequentially dispensing, methods for producing a sequence of allergen doses with reduced adverse reactions, methods for producing a stock solution for dilution, and methods for producing cooperative pre-filled vials are also disclosed.

### CROSS-REFERENCE TO RELATED APPLICATIONS

This application is a Continuation-in-Part of U.S. patent application Ser. No. 13/352,277, filed Jan. 17, 2012, the entire content of which is hereby incorporated by reference, and which is a divisional of U.S. application Ser. No. 12/981,726, filed on Dec. 30, 2010.

### FIELD OF THE INVENTION

This invention relates to sublingual immunotherapy (SLIT), and particularly to preparations, methods, and delivery systems for sublingual immunotherapy.

### BACKGROUND OF THE INVENTION

Sublingual Immunotherapy is a form of immunotherapy that involves placing drops of allergen extract under the tongue. The allergen is administered in incrementally increasing amounts, starting at a very dilute allergen concentration and at a low volume of the allergen solution. Once higher concentration and higher volume is well tolerated, the patient continues taking the immunotherapy at a maintenance dose. Some people refer to this process as “allergy drops,” because Sublingual Immunotherapy is an alternative to “allergy shots”. Sublingual Immunotherapy has been used for years in Europe, and recently has been increasingly used in the United States. In Europe, Sublingual Immunotherapy has substantially replaced allergy shots, and it probably will do the same in the USA.

Sublingual Immunotherapy is usually delivered in one of two ways: as drops of allergen extract, or as tablets of allergen extract, both being placed under the tongue, and then either swallowed or expectorated.

Generally, Sublingual Immunotherapy is administered daily, or multiple times per week, over a period of years. Most patients are able to self-administer Sublingual Immunotherapy at home. Studies have looked at giving Sublingual Immunotherapy before pollen season, during pollen season, before and during pollen season, or all year-round. It is not yet known what the best dosing regimen is for Sublingual Immunotherapy.

Over the past 10 years, the safety of Sublingual Immunotherapy has been well documented. The most common uncomfortable side effects of sublingual immunotherapy are oral itchiness (pruritus), lip itchiness, oral swelling, tongue swelling, and throat irritation. These side effects can cause discomfort such that a patient may not properly comply with treatment, which can slow the progression of the dose, thereby limiting the efficacy of the treatment, or can result in the patient refusing the treatment entirely.

### SUMMARY OF THE INVENTION

One general aspect of the invention is a preparation of a sublingual immunotherapy allergen cooperative with a mast cell stabilizer, and possibly also cooperative with at least one additional allergy symptom reducer, selected from the group including: antihistamines and leukotriene inhibitors. Examples of mast cell stabilizers include: Cromolyn sodium (cromoglicic acid) 4-200 mg/ml and 4-200 mg, Olopatatidine 1 mg/ml and 5 mg, Nedocromil 20-40 mg/ml, Ketotifen 0.25 mg/ml and 1 mg, and Quercetin.

Examples of antihistamines include: loratidine, diphenhydramine, fexofenadine, azelastine, and cetirizine. Examples of leukotriene inhibitors include montelukast. The allergen can be mixed into a Cromolyn sodium solution, for example, and the mixture can be applied sublingually. Alternatively, an allergen powder can be mixed with a mast cell stabilizer powder, and optionally at least one allergy-symptom-reducer powder, and the powder mixture can be included in a sublingual dissolving tablet.

Another embodiment is a bilayer sublingual dissolving tablet for providing a dose of an allergen (or combination of allergens), along with a mast cell stabilizer to prevent or reduce uncomfortable itchiness, where a first layer of the tablet contains the allergen, and the second layer of the tablet contains the mast cell stabilizer, and possibly an antihistamine and/or a leukotriene inhibitor.

Another embodiment is a tri-layer sublingual dissolving tablet for providing a dose of an allergen (or combination of allergens), along with a mast cell stabilizer to prevent or reduce uncomfortable oral itchiness, the tablet having an inner layer disposed between two outer layers, where the inner layer contains the allergen, and the two outer layers contain a mast cell stabilizer, and possibly an antihistamine and/or a leukotriene inhibitor. This embodiment allows the mast cell stabilizer to substantially prevent an oral allergic reaction to the allergen. Optionally, by making the inner layer smaller in diameter than the two outer layers, and coating the entire tri-layer structure with a sublingually dissolving coating that can include flavoring, the inner layer of allergen will be dissolved substantially after the mast cell stabilizer, thereby enhancing the allergy symptom reduction/prevention effect of the mast cell stabilizer.

Another embodiment includes a pair of squeezable ampoules, a first squeezable ampoule containing a dose of an allergen solution, and a second squeezable ampoule containing a dose of a mast cell stabilizer for reducing or preventing an oral allergic reaction to the allergen, the mast cell stabilizer being optionally mixed with an antihistamine and/or a leukotriene inhibitor. The two squeezable ampoules are then squeezed in tandem so the two solutions together make contact under the tongue and begin to mix on contact. The two squeezable ampoules can be enclosed in a flexible sleeve so that when the flexible sleeve is squeezed, both squeezable ampoules inside are squeezed at substantially the same time, and to substantially the same extent.

Another embodiment includes a pair of squeezable ampoules, a first squeezable ampoule containing a dose of an allergen solution, and a second squeezable ampoule containing a dose of a mast cell stabilizer solution for reducing or preventing an oral allergic reaction to the allergen, the mast cell stabilizer being optionally mixed with an antihistamine and/or a leukotriene inhibitor. Each ampoule is sealed with a pressure-break seal. The pressure required to break the seal of the ampoule containing the mast cell stabilizer (that actually functions substantially as an allergy symptom preventer) is less than the pressure to break the seal of the ampoule containing the allergen, Thus, a slow firm pressure applied to the joined pair of squeezable ampoules will first break the seal of the squeezable ampoule containing the mast cell stabilizer solution, thereby first administering the mast cell stabilizer solution, and then will break the seal of the squeezable ampoule containing the allergen solution. Thus, the sublingual presence of the mast cell stabilizer solution before the sublingual contact of the allergen solution will substantially prevent uncomfortable allergic symptoms, such as oral itchiness. To ensure that the two seals break under different pressure, and therefore at different times, the seals can be made to be of different thickness, for example. Or the two seals can be of different diameters and be of the same thickness.

Or the two seals can be of the same diameter and of the same thickness, but the contents of the squeezable ampoule containing the mast cell stabilizer solution will be directly ejected via a short channel to an opening, while the contents of the squeezable ampoule containing the allergen solution will be circuitously ejected via a channel at least running along the length of the ampoule before it is ejected through an opening. Thus, a steady squeeze of both ampoules will first eject the mast cell stabilizer, and will later eject the allergen, allowing the mast cell stabilizer to substantially reduce or prevent an oral allergic reaction to the allergen when it eventually makes contact with the sublingual oral mucosa.

Another general aspect of the invention is a delivery system including an ordered set of squeezable ampoules, each squeezable ampoule of the ordered set having a quantity of allergen in accordance with a monotonically increasing dilution sequence, such that each successive squeezable ampoule in the ordered set will administer an amount of allergen that is equal to or greater than the amount of allergen in the previous squeezable ampoule in the ordered set. Each squeezable ampoule in the ordered set is paired with a second squeezable ampoule containing a dose of a mast cell stabilizer for reducing or preventing an oral allergic reaction to the allergen, the mast cell stabilizer being optionally mixed with an antihistamine and/or a leukotriene inhibitor. The dose of the mast cell stabilizer of the second squeezable ampoule of each pair of ampoules remains constant as the amount of allergen in each corresponding squeezable ampoule of the ordered set monotonically increases with each successive squeezable ampoule of allergen. The two squeezable ampoules in a pair of ampoules are squeezed in tandem so the two solutions together make contact under the tongue and begin to mix on contact. The two squeezable ampoules can be enclosed in a flexible sleeve so that when the flexible sleeve is squeezed, both squeezable ampoules inside are squeezed at substantially the same time, and to substantially the same extent. The squeezable ampoule of each pair can be configured to release the dose of mast cell stabilizer before the squeezable ampoule containing the allergen releases the allergen solution.

Another general aspect of the invention is a delivery system including an ordered set of fast-dissolving sublingual tablets, each fast-dissolving sublingual tablet of the ordered set having a quantity of allergen in accordance with a monotonically increasing quantity of allergen, such that each successive fast-dissolving sublingual tablet in the ordered set will administer an amount of allergen that is equal to or greater than the amount of allergen in the previous fast-dissolving sublingual tablet in the ordered set. Each fast-dissolving sublingual tablet in the ordered set is paired with a second fast-dissolving sublingual tablet containing a dose of a mast cell stabilizer for reducing or preventing an oral allergic reaction to the allergen, the mast cell stabilizer being optionally mixed with an antihistamine and/or a leukotriene inhibitor. The dose of the mast cell stabilizer of the second fast-dissolving sublingual tablet of each pair of tablets remains constant as the amount of allergen in each corresponding fast-dissolving sublingual tablet of the ordered set monotonically increases with each successive tablet of allergen. The two tablets in a pair of tablets can be placed under the tongue together, or the mast cell stabilizer tablet can be placed first, and when dissolved, followed by the allergen tablet.

Another general aspect of the invention is a delivery system including an ordered set of bilayer fast-dissolving sublingual tablets, each bilayer fast-dissolving sublingual tablet of the ordered set including a first layer having a quantity of allergen in accordance with a monotonically increasing quantity of allergen, such that each successive bilayer fast-dissolving sublingual tablet in the ordered set will administer an amount of allergen that is equal to or greater than the amount of allergen in the previous fast-dissolving sublingual tablet in the ordered set. Each fast-dissolving sublingual tablet in the ordered set also includes a second layer containing a dose of a mast cell stabilizer for reducing or preventing an oral allergic reaction to the allergen in the first layer, the mast cell stabilizer being optionally mixed with an antihistamine and/or a leukotriene inhibitor. The dose of the mast cell stabilizer of the second layer remains constant as the amount of allergen in the first layer monotonically increases with each successive bilayer tablet.

Another general aspect of the invention is a delivery system including an ordered set of coated fast-dissolving sublingual tablets, each coated fast-dissolving sublingual tablet of the ordered set including a center having a quantity of allergen in accordance with a monotonically increasing quantity of allergen, such that each successive coated fast-dissolving sublingual tablet in the ordered set will administer an amount of allergen that is equal to or greater than the amount of allergen in the previous fast-dissolving sublingual tablet in the ordered set. Each coated fast-dissolving sublingual tablet in the ordered set also includes a coating containing a dose of a mast cell stabilizer for reducing or preventing an oral allergic reaction to the allergen in the center, the mast cell stabilizer being optionally mixed with an antihistamine and/or a leukotriene inhibitor. The dose of the mast cell stabilizer in the coating remains constant as the amount of allergen in the center monotonically increases with each successive coated tablet. in the ordered set.

Another general aspect of the invention is a delivery system including an ordered set of tri-layer fast-dissolving sublingual tablets, each tri-layer fast-dissolving sublingual tablet of the ordered set including an inner layer having a quantity of allergen in accordance with a monotonically increasing quantity of allergen, such that each successive tri-layer fast-dissolving sublingual tablet in the ordered set will administer an amount of allergen that is equal to or greater than the amount of allergen in the previous tri-layer fast-dissolving sublingual tablet in the ordered set. Each fast-dissolving sublingual tablet in the ordered set also includes two outer layers together containing a dose of a mast cell stabilizer for reducing or preventing an oral allergic reaction to the allergen in the inner layer, the mast cell stabilizer being optionally mixed with an antihistamine and/or a leukotriene inhibitor. The dose of the mast cell stabilizer provided by the two outer layers remains constant as the amount of allergen in the inner layer monotonically increases with each successive tri-layer tablet.

In some embodiments, an ordered set of fast-dissolving sublingual tablets is packaged in a sequential blister pack, such that the ordered set of tablets can easily be taken in proper sequence so as to provide a monotonically increasing amount of allergen, along with a consistent level of mast cell stabilizer that can be incorporated into each tablet, or can be provided as a separate companion oral-allergy-symptom-reducing tablet for each allergen tablet.

In some embodiments, an ordered set of fast-dissolving sublingual tablets is pre-loaded into a spring-loaded plastic magazine, such that the ordered set of fast-dissolving sublingual tablets can easily be taken in proper sequence off the top opening of the spring-loaded plastic magazine, so as to provide a monotonically increasing amount of allergen, along with a consistent level of mast cell stabilizer that can be included with the allergen as a bi-layer tablet, tri-layer tablet, or coated tablet, or can be provided as a pair of tablets dispensed together, one tablet providing the allergen and a companion tablet providing the mast cell stabilizer. Each spring-loaded magazine can contain seven or fourteen doses of allergen, for example, providing one or two doses per day, respectively, for a week.

In some embodiments, an ordered set of squeezable ampoules is packaged in a spring-loaded plastic magazine, such that the ordered set of squeezable ampoules can easily be taken in proper sequence off the top opening of the spring-loaded plastic magazine so as to provide a monotonically increasing amount of allergen, along with a consistent level of mast cell stabilizer that can be mixed with the allergen in each ampoule, or can be provided as a joined pair of ampoules each having an allergen ampoule and a mast cell stabilizer ampoule. Each spring-loaded magazine can contain for example seven doses of allergen, i.e., one ampoule per day (or one ampoule pair per day), or fourteen doses, i.e., two doses per day. The monotonically increasing amount of allergen can be provided as a monotonically increasing amount of allergen at a fixed concentration, or as a monotonically increasing concentration of allergen in a fixed amount of solution.

The preparation of the invention can be used sublingually to provide sublingual immunotherapy with reduced or substantially no localized oral itchiness, as compared with the localized oral itchiness that is often associated with sublingual immunotherapy, thereby enhancing patient compliance. The mixing of an allergen used for immunotherapy in a mast cell stabilizer solution, such as Cromolyn sodium solution, is likely to be effective for both pollen allergens, and non-pollen allergens such as dust mite, mold, and pet dander (e.g., cat, dog), as well as for insect venom, and for the new food desensitization protocols. The invention provides decreased local side effects of sublingual immunotherapy by providing a mast cell stabilizer, such as Cromolyn, along with an allergen. The mast cell stabilizer can be mixed with an antihistamine and/or a leukotriene inhibitor.

Another aspect of the invention is Oral Immunotherapy (OIT) with reduced gastrointestinal discomfort, such as reduced abdominal pain, reduced diarrhea, and reduced vomiting. In OIT, an allergen is micro-encapsulated in an enteric coated bead or beads, which pass(es) through the stomach intact, and then dissolve(es) in the small intestine based on a pH-dependent effect, thereby releasing the allergen to make contact with the mucosal surface of the small intestine. By also microencapsulating a mast cell stabilizer such as Cromolyn, and swallowing the micro-encapsulated Cromolyn along with the micro-encapsulated allergen, the gastrointestinal discomfort can be reduced or prevented. A dose of 100 mg to 200 mg of Cromolyn would be effective. Alternatively, swallowing Cromolyn solution, at a concentration of 100 mg/5 ml to 200 mg/5 ml, along with the micro-encapsulated allergen would reduce or avoid the unpleasant gastrointestinal effects typically encountered in OIT.

A general aspect of the invention is a preparation for sublingual immunotherapy, wherein the preparation includes a mast cell stabilizer; and at least one allergen suitable for sublingual immunotherapy.

In some embodiments, the allergen or combination of allergens includes at least one of: grass pollen, tree pollen, ragweed pollen, weed pollen, dust mite, cat allergen, dog allergen, horse allergen, cow allergen, mice allergen, bird allergen, rabbit allergen, mold spores, hymenoptera venom, cockroach allergen, milk allergen, egg allergen, peanut allergen, nut allergen, seed allergen, wheat allergen, soy allergen, and latex.

In some embodiments, the mast cell stabilizer is Cromolyn sodium.

In some embodiments, the preparation further includes a diluent. In further embodiments, the diluent is selected from: sterile water, glycerin.

In some embodiments, the preparation further includes: at least one of an antihistamine, a leukotriene inhibitor. In further embodiments, the antihistamine is selected from: diphenhydramine, cetririzine, azelestine, ketotifen, loratidine, olopatatidine, fexofenadine. In other further embodiments, the leukotriene inhibitor is montelukast.

In some embodiments, the at least one allergen is diluted in a diluent containing a concentration of Cromolyn at 4-200 mg/ml.

Another general aspect of the invention is a multi-layer tablet preparation for sublingual immunotherapy. This preparation includes a first tablet layer including a mast cell stabilizer; and a second tablet layer including an allergen suitable for sublingual immunotherapy.

In some embodiments, the first tablet layer is fast-dissolving such that the first tablet layer dissolves faster than the second tablet layer so that the mast cell stabilizer is rapidly delivered to the sublingual mucosa before most of the allergen makes contact therewith.

Another general aspect of the invention is a multi-layer tablet preparation for sublingual immunotherapy, this preparation including a first tablet layer including a mast cell stabilizer; a second tablet layer including an allergen suitable for sublingual immunotherapy; and a third tablet layer including a mast cell stabilizer.

In some embodiments, the preparation further includes a dissolvable coating fully enclosing the first, second, and third layers, and wherein the second tablet layer is of a smaller diameter than the first tablet layer and the third tablet layer, so as to ensure that the second layer begins dissolving after the first layer and third layer have started to dissolve.

In some embodiments, the first layer joins with the third layer so as to enclose the second layer.

Another general aspect of the invention is a tablet preparation for sublingual immunotherapy, this preparation including a tablet center including an allergen suitable for sublingual immunotherapy; and a tablet coating, fully enclosing the tablet center, the tablet coating including at least a mast cell stabilizer.

Another general aspect of the invention is an apparatus for administration of sublingual immunotherapy, this apparatus including a first squeezable ampoule of mast cell stabilizer solution; and a second squeezable ampoule of an allergen solution.

In some embodiments, the apparatus further includes a flexible sleeve surrounding the first squeezable ampoule and the second squeezable ampoule, so that when the flexible sleeve is squeezed, the first and second squeezable ampoules are squeezed at substantially the same time, and to substantially the same extent.

In some embodiments, each ampoule is sealed with a pressure-break seal. In further embodiments, the pressure required to break the seal of the first squeezable ampoule containing the mast cell stabilizer is less than the pressure required to break the seal of the squeezable ampoule containing the allergen, such that simultaneously squeezing both ampoules will first break the seal of the first squeezable ampoule containing the mast cell stabilizer, thereby first administering the mast cell stabilizer, and then will break the seal of the second squeezable ampoule containing the allergen solution.

In some embodiments, the thickness of the seal of the first squeezable ampoule is less than the thickness of the second squeezable ampoule, such that the two seals break under different pressure, and therefore at different times.

Another general aspect of the invention is an apparatus for sublingual immunotherapy, this apparatus including: a first ampoule of a solution of mast cell stabilizer; a second ampoule of a solution of an allergen; and a mixing nozzle in fluid communication with a pressure seal of the first ampoule and a pressure seal of the second ampoule, such that upon squeezing the first and second ampoules with a pressure sufficient to break both pressure seals, the solution of mast cell stabilizer and the solution of the allergen are ejected into the mixing nozzle, thereby mixing the mast cell stabilizer with the allergen prior to flowing out of the mixing nozzle onto the sublingual mucosa.

Another general aspect of the invention is a method for administration of sublingual immunotherapy, this method including: applying sublingually a sequence of allergen doses of monotonically increasing amount; and applying sublingually, for each allergen dose of the sequence, a constant dose of mast cell stabilizer.

In some embodiments, the constant dose of mast cell stabilizer is applied sublingually just prior to each dose of the sequence of allergen doses.

In some embodiments, the constant dose of mast cell stabilizer is applied sublingually with each dose of the sequence of allergen doses.

Another general aspect of the invention is a method for performing a dilution sequence of an allergen to produce a sequence of allergen doses of increasing concentration, each allergen dose including a mast cell stabilizer dose at a constant concentration, this method including: obtaining a stock solution of an allergen at a first concentration mixed with a mast cell stabilizer at a selected concentration; obtaining a first pre-measured vial containing a diluent mixed with the mast cell stabilizer at the selected concentration; diluting a portion of the stock solution with the contents of the first pre-measured vial to produce a first-dilution solution of the allergen mixed with the mast cell stabilizer at the selected concentration; obtaining a second pre-measured vial containing the diluent mixed with the mast cell stabilizer at the selected concentration; and diluting a portion of the first-dilution solution with the contents of the second pre-measured vial to produce a second-dilution of the allergen mixed with the mast cell stabilizer at the selected concentration.

In some embodiments, the mast cell stabilizer is Cromolyn, and the selected concentration is 4-200 mg/ml.

In some embodiments, the diluent is at least one of: glycerin and water.

Another general aspect of the invention is a stock solution of an allergen at a stock concentration mixed with Cromolyn at a concentration of 4-200 mg/ml.

Another general aspect of the invention is a pre-filled vial of sterile water or glycerine having Cromolyn dissolved therein at a concentration of 4-200 mg/ml for use as a diluent of a stock solution also having the Cromolyn dissolved therein at a concentration of 4-200 mg/ml, the pre-filled vial being usable in a dilution sequence for sublingual immunotherapy.

In some embodiments, the pre-filled vial is prefilled to a volume selected from: 9 ml, 4.5 ml, 1.8 ml.

Another general aspect of the invention is a dosing system for providing sublingual immunotherapy via sublingual tablets, the dosing system including: a therapeutic sequence of sublingual tablets, the therapeutic sequence of sublingual tablets having a monotonically increasing dose of allergen, and a same dose of mast cell stabilizer in each tablet of the therapeutic sequence; and a plurality of maintenance dose sublingual tablets, each maintenance dose sublingual tablet having a same maintenance dose of allergen, and the same dose of mast cell stabilizer as in each sublingual tablet of the therapeutic sequence.

In some embodiments, each sublingual tablet includes a dose of Cromolyn of 4-200 mg.

In some embodiments, each sublingual tablet dissolves within 5 to 120 seconds.

In some embodiments, each sublingual tablet can be taken soon after taking a preventative tablet of Cromolyn of 4-200 mg to prevent an uncomfortable oral allergic reaction to the allergen.

In some embodiments, each sublingual tablet can be followed promptly by a therapeutic tablet of Cromolyn of 4-200 mg to treat an uncomfortable oral allergic reaction to the allergen.

Another general aspect of the invention is a dosing system for providing sublingual immunotherapy via sublingual ampoules, this dosing system including: a therapeutic sequence of sublingual ampoules, the therapeutic sequence of sublingual ampoules having a monotonically increasing dose of allergen, and a same dose of mast cell stabilizer in each ampoule of the therapeutic sequence; and a plurality of maintenance dose sublingual ampoules, each maintenance dose sublingual ampoule having a same maintenance dose of allergen, and the same dose of mast cell stabilizer as in each sublingual ampoule of the therapeutic sequence.

In some embodiments, the therapeutic sequence of sublingual ampoules is contained in a spring-loaded magazine so that the therapeutic sequence of sublingual ampoules can be dispense in sequence.

Another general aspect of the invention is an apparatus for sequentially dispensing a therapeutic sequence of sublingual ampoules, this apparatus including: a spring-loaded magazine for dispensing joined pairs of squeezable ampoules in a sequence having monotonically increasing dose of allergen and constant dose of mast cell stabilizer; and a stack of joined pairs of squeezable ampoules in a sequence having monotonically increasing dose of allergen and constant dose of mast cell stabilizer.

Another general aspect of the invention is a method for sublingual immunotherapy, this method including: taking a sequence of allergen tablets of monotonically increasing dose; and taking a constant sublingual dose of cromolyn 4-200 mg along with each allergen tablet of the sequence.

In some embodiments, the constant sublingual dose of cromolyn 4-200 mg is taken in the form of: sublingual tablet; liquid; spray; and drops.

### BRIEF DESCRIPTION OF THE DRAWINGS

The invention will be more fully understood by reference to the detailed description, in conjunction with the following figures, wherein:

FIG. 1 is a side view of a bi-layer tablet having a layer of allergen, and a layer of mast cell stabilizer;

FIG. 2 is a side view of a tri-layer tablet having a middle layer of allergen, and two outer layers of mast cell stabilizer;

FIG. 3 is a side view of a coated tri-layer tablet, the coating ensuring that the outer layers begin dissolving before the middle layer begins dissolving;

FIG. 4 is a side view of a single layer coated tablet having an allergen center and a mast cell stabilizer coating;

FIG. 5 is a top view of a joined pair of squeezable ampoules having different pressure-break seals;

FIG. 6 is a view along A of FIG. 5 of the joined pair of squeezable ampoules, also showing a surrounding flexible sleeve;

FIG. 7 is a top view of a joined pair of squeezable ampoules having same pressure-break seals, and a different channel length to each respective ampoule opening; and

FIG. 8 is a perspective view of a spring-loaded plastic magazine for sequentially dispensing joined pairs of squeezable ampoules so as to provide a sequence of monotonically increasing doses of allergen and a constant dose of mast cell stabilizer.

### DETAILED DESCRIPTION

The mast cell stabilizer Cromolyn is a calcium channel blocker that interferes with the release of mediators from mast cells, including histamines and leukotrienes. In one embodiment of the invention, an allergen (such as pollen, dust mites, mold, cockroach allergens, cat allergens, or dog allergens, food allergens (such as peanut)) is dissolved in a Cromolyn sodium solution containing Cromolyn 100-200 mg per 5 ml of sterile water or glycerine. In preliminary studies, this concentration of Cromolyn solution has been shown by the allergist Ziv Harish MD to prevent and/or treat oral itchiness associated with ingestion of raw fruits and vegetables that cross-react with pollen. Combining sublingual administration of a Cromolyn solution containing Cromolyn 20-1000 mg per 5 ml of sterile water or glycerine, along with allergens administered in sublingual immunotherapy will prevent or rapidly diminish the oral itchiness typically associated with sublingual immunotherapy. Alternatively, combining sublingual administration of Cromolyn powder 4-200 mg along with allergen powder administered in sublingual immunotherapy will prevent or rapidly diminish the oral itchiness typically associated with sublingual immunotherapy.

A preparation for sublingual immunotherapy can be made by mixing an allergen suitable for sublingual immunotherapy with a mast cell stabilizer, such as Cromolyn, the mast cell stabilizer substantially reducing or eliminating the symptoms of oral itchiness and/or lip itchiness that many patients experience when treated with sublingual immunotherapy.

Cromolyn sodium has been found to have an extremely good safety record, showing no toxicity at high doses. Cromolyn does not interfere with or react with proteins, such as the proteins found in the allergens used in sublingual immunotherapy.

An amount of allergen can be added to a solution of Cromolyn sodium without concerns of changing the solubility or the potency of the allergen, or of the Cromolyn sodium. Likewise, an amount of allergen powder can be added to an amount of Cromolyn powder without concerns of affecting the potency of the allergen, or the potency of the Cromolyn.

Quercetin is a mast cell stabilizer, and can be used to reduce oral itchiness of allergens used in sublingual immunotherapy, as long as the quercetin is administered sublingually before sublingual administration of the allergen, according to Weng, et al, “Quercetin Is More Effective than Cromolyn in Blocking Human Mast Cell Cytokine Release and Inhibits Contact Dermatitis and Photosensitivity in Humans”, Weng Z, Zhang B, Asadi S, Sismanopoulos N, Butcher A, et al. (2012). PLoS ONE 7(3): e33805. doi:10.1371/journal.pone.0033805.

Sublingual Allergy Immunotherapy induces tolerance to an allergen (or mixture of allergens) via a series of incrementally increasing dose of the allergen (or allergens), administered sublingually. Different allergens have different allergenic reactivity, and thus the clinical potency of an allergen extract cannot always be determined by the concentration of the allergen in the solution. Some allergen extract potency is expressed in AU (Allergy Units) per milliliter (ml), in BAU (Bioequivalent Allergy Units) per ml or, IR (Index of Reactivity). In some cases, the amount of the allergen is expressed in PNU (Protein Nitrogen Units), which are used to specify the amount of protein in the solution, or by a weight/volume ratio (weight of the allergen per unit volume of the diluent—usually in micrograms per milliliter (p/ml). Another unit is SQ-T; it is a unit comparing the amount of allergen given orally or sublingually as the maintenance allergen dose administered subcutaneously every two to four weeks in traditional allergy immunotherapy (which is generally 20-200 times greater for the former routes).

An effective maintenance dose (units) of an allergen depends on the specific allergen. Examples of maintenance units for some common environmental allergens include: Rye grass: 25000-50000 PNU; D. Farinae (dust mite): 3750-7500 AU; and Cat: 3750-7500 BAU. Commercially available sublingual tablet ORALAIR™ provides a five-grass pollen mixture. The tablets are available in two strengths: 100 IR and 300 IR. Patients are instructed to take one tablet of 100 IR for one day, then two 100 IR tablets on the second day, and then one 300 IR tablet each day throughout the season. Studies were also conducted with grass tabs containing 25,000-1 million SQ-T units which contain 5-200 mcg of the grass allergenic protein (Khinchi M S et al. Clinical efficacy of sublingual and subcutaneous birch pollen allergen-specific immunotherapy: a randomized, placebo-controlled, double blind, double-dummy study. Allergy 2004: 59:45)

Maintenance solution strength varies for different allergens from 20 drops of the ragweed 100 IR/ml solution to 20 drops of the 300 IR solution for dust mites (D. Farinae and D. Pteronyssinus). To achieve the maintenance dose, patients are instructed to start with one drop, and gradually climb to 20 drops a day. Of note is that generally the increments of the doses are done daily, but there are rush protocols instructing patients to take doses of 1, 5, 10, and 20 drops 30 minutes apart.

Thus, to arrive at the maintenance dose, a progressively increasing amount of allergen is used by starting at a very small starting dose of allergen at a concentration of 1:1000 of the maintenance dose, and gradually increasing the number of drops of the allergen solution up to 1 ML before switching to a 1:100 concentration solution. Then again, the number of drops will be gradually increased before switching to a more concentrated solution, and so forth.

Generally, sublingual immunotherapy is started with sublingual administration of a very small volume of a very dilute allergen. The dose is gradually increased by increasing the amount of the allergen up to a maintenance dose that is administered periodically, the maintenance dose possibly being 1000 or more times more concentrated than the concentration of the allergen in the initial dose.

Allergists typically obtain the allergenic extracts from special laboratories that purify and quantify the allergen, and then dissolve it in a sterile diluent, such as sterile water or sterile glycerin. The resulting solution is commercially available as a “stock solution” that is concentrated to an extent that is useful for creating a therapeutic dilution sequence, and then for creating a customized regular maintenance dose.

To treat an allergy patient, an allergist selects and obtains an appropriate stock solution, and then dilutes the stock solution to create the first (lowest potency) therapeutic sublingual dose. The dilution is often made serially by taking a small volume of the stock solution, and then mixing it in a larger volume of diluent to create a once-diluted extract. Then, the allergist takes the same small volume of the resulting once-diluted extract, and further mixes it in another same larger volume of diluent to create a twice-diluted extract. This dilution process is repeated until the desired concentration of allergen (as determined by the allergist) is achieved.

For example, to make a dose of cat allergen at a concentration of 1 unit/ml, one can take 1 ml of 10,000 units/ml stock solution, and mix it in 9 ml of diluent to make 10 ml of once-diluted extract at a concentration of 1,000 units/ml of cat allergen. By mixing 1 ml of this once-diluted solution in 9 ml of diluent, a twice-diluted extract at a concentration of 100 units/ml will be obtained. Repeating this procedure two more times will produce an extract at a concentration of 1 unit/ml.

Vials of diluent, pre-filled to selected volumes, are commercially available to ease ten-fold dilution sequences. The vials are available prefilled with volumes of 9 ml, 4.5 ml, and 1.8 ml of diluent so as to facilitate dilution sequences that involve mixing 1 ml, 0.5 ml, or 0.2 ml of progressively diluted allergen solution respectively with 9 ml, 4.5 ml, and 1.8 ml of diluent. The choice of volume is determined by the amount needed by the allergist in the context of his/her particular practice.

According to the invention, we teach including a mast cell stabilizer, such as Cromolyn, along with a diluent such as sterile water or glycerine, so as to provide a reduced-allergic-symptom diluent contained in each pre-filled vial. The presence of a mast cell stabilizer (such as Cromolyn) at an effective fixed concentration serves to reduce or even prevent local allergic reactions to sublingual contact (as well as other oral contact) with the allergen that is introduced sublingually at progressively higher concentrations during sublingual immunotherapy.

To ease the process of dilutions: The stock solution of the invention is the concentrated allergen solution from which the allergen solution dilutions are made, and will also contain a fixed concentration of mast cell stabilizer. The diluent of the invention is the solution with which the stock solution of the invention will be diluted, and will contain the same fixed concentration of mast cell stabilizer as in the stock solution, dissolved in a pre-measured volume of sterile water or glycerine, the diluent of the invention being contained in pre-filled vials.

Note that the concentration of Cromolyn in each dilution of a dilution sequence of the allergen remains the same over the dilution sequence using the reduced-allergic-symptom diluent including Cromolyn as the diluent.

Also according to the invention, the stock solution of an allergen includes a mast cell stabilizer, such as Cromolyn, at a concentration of the mast cell stabilizer that is effective to substantially reduce or even prevent a local allergic reaction to sublingual administration of a solution of the allergen mixed with mast cell stabilizer at each step of the dilution sequence.

Pre-filled diluent vials of the invention contain the same concentration of Cromolyn as that of the concentration of Cromolyn in the stock solution that contains the allergen dissolved in Cromolyn solution. Thus, for example, starting with 1 ml of a stock solution containing 1000 units of an allergen and 20 mg of Cromolyn (a Cromolyn concentration of 20 mg/ml), and transferring it to 9 ml of diluent containing 180 mg (9 ml times 20 mg/ml) of Cromolyn, will result in a solution containing 100 units of allergen per ml, and again a Cromolyn concentration of 20 mg/ml.

The Cromolyn concentration found to be effective is based on the concentration of 20 mg/ml to 40 mg/ml found by the allergist Ziv Harish, MD to be effective for the prevention and treatment of Oral Allergy Syndrome (OAS), and therefore the concentration for sublingual immunotherapy falls generally within a range of 4 mg/ml to 200 mg/ml for a 1 ml dose delivered sublingually, for example, depending on the particular patient, and the judgment of the allergist.

The stock solution of the invention and the pre-filled diluent vials of the invention can also include a supporting fixed concentration of an antihistamine (1-10 mg/ml of antihistamine, such as Zyrtek™ (cetirizine) or Claritin™ (loratidine)), and/or a leukotriene inhibitor (1-10 mg/ml Singulair™ montelukast) along with the mast cell stabilizer to enhance the effect of the mast cell stabilizer.

The dissolvable Cromolyn tablets can contain 4-200 mg of Cromolyn, such as a 4 mg dissolvable Cromolyn tablet for children, 100 mg tablet for adults, and 200 mg tablet for adults extra-strength version.

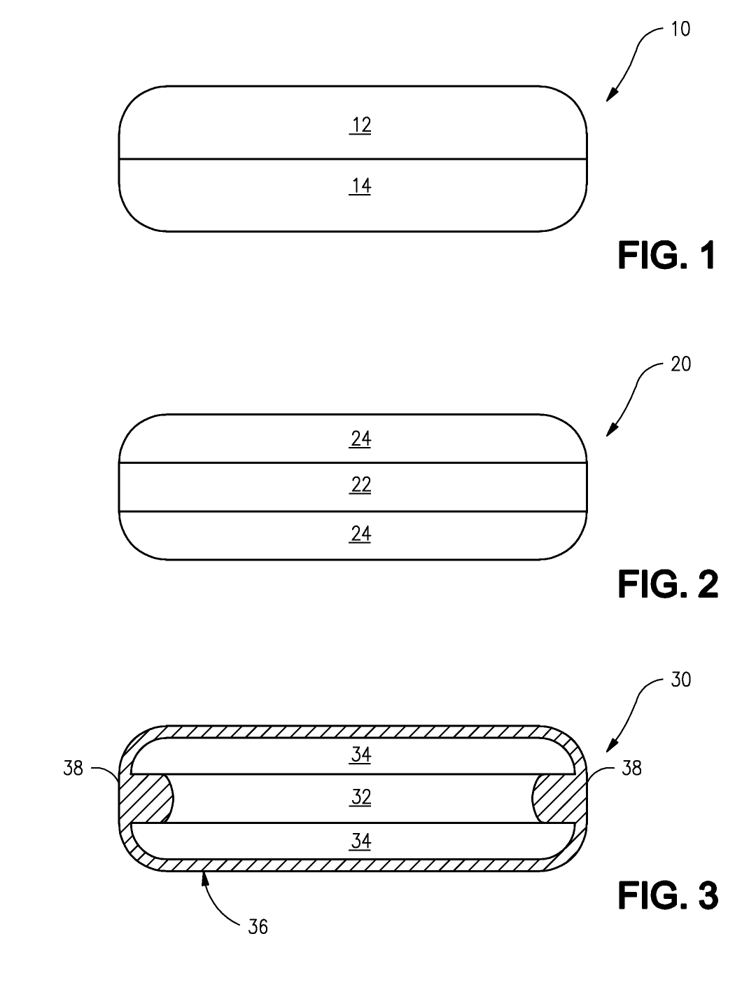

Referring to FIG. 1, a bi-layer tablet 10 has a top layer 12 and a bottom layer 14. One layer can include a dose of allergen, and the other layer can include a dose of a mast cell stabilizer, such as Cromolyn. Either layer can also include an antihistamine and/or a leukotriene inhibitor. Flavoring can be included. The tablet can also include ingredients that facilitate rapid dissolving sublingually, as are well-known in the art. The top layer 12 can have ingredients that causes it to dissolve FASTER than the ingredients that cause the bottom layer 14 to dissolve. For example, the top layer 12 can be configured to dissolve within 5 to 20 seconds so as to release the mast cell stabilizer rapidly and before the bottom layer dissolves, while the bottom layer 14 can be configured to dissolve within 30 to 60 seconds so as to release the allergen substantially AFTER the mast cell stabilizer has affected the local oral mucosa. The differential dissolution rate helps the mast cell stabilizer to take effect before a substantial amount of allergen makes contact with the oral mucosa, thereby limiting or even preventing uncomfortable adverse reactions to the allergen. A sequence of bi-layer tablets for sublingual immunotherapy according to the invention includes a sequence of doses of allergen that monotonically increases as one progresses through the sequence, while the dose of mast cell stabilizer, and possibly other allergic symptom reducer (antihistamine and/or leukotriene inhibitor) remains the same over the sequence of bi-layer tablets.

Referring to FIG. 2, a tri-layer tablet 20 has a middle layer 22, and two outer layers 24. The middle layer 22 includes a dose of allergen, and the two outer layers together include a dose of a mast cell stabilizer, such as Cromolyn. Any of the layers 22, 24 can also include an antihistamine and/or a leukotriene inhibitor. Flavoring can be included in any layer to enhance patient compliance. The tablet can also include ingredients that facilitate rapid dissolving sublingually, as are well-known in the art. The outer layers 24 can have ingredients that cause them to dissolve FASTER than the ingredients that cause the middle layer 22 to dissolve. For example, the outer layers 24 can be configured to dissolve within 5 to 20 seconds, while the middle layer 22 can be configured to dissolve within 30 to 60 seconds. The differential dissolution rate helps the mast cell stabilizer to take effect before a substantial amount of allergen makes contact with the oral mucosa, thereby limiting or even preventing uncomfortable adverse reactions to the allergen. A sequence of tri-layer tablets for sublingual immunotherapy according to the invention includes a dose of allergen that monotonically increases as one progresses through the sequence of tablets, while the dose of mast cell stabilizer, and possibly a dose of another allergic symptom reducer (antihistamine and/or leukotriene inhibitor) remains the same over the sequence of bi-layer tablets.

Referring to FIG. 3, a coated tri-layer tablet 30 having a recessed middle layer 32 sandwiched between two outer layers 34 is also coated by a coating layer 36 that fills in a recess 38 surrounding the recessed middle layer 32. The coating layer 36 includes ingredients that facilitate rapid dissolving sublingually, as are well-known in the art. Due to the recess 38, being thicker than the rest of the layer 36, the layers 34 will be exposed to saliva before the middle layer 32. According to the invention, the layers 34 contain a mast cell stabilizer, such as Cromolyn, and the middle layer 32 contains a dose of allergen that can cause uncomfortable adverse reactions when contacting unmedicated oral mucosa. However, since the outer layers 34 will dissolve BEFORE the middle layer 32 is exposed to saliva, and therefore before the oral mucosa is exposed to the allergen, the mast cell stabilizer can reduce, and possibly even PREVENT an uncomfortable adverse reaction, such as pruritis, swelling (edema), or other irritation of the lips, tongue, mouth, and throat. The layers 32 and 34 can also be rapidly-dissolving (within 5 to 60 seconds) to ensure that the full dose of both allergen and mast cell stabilizer are substantially delivered sublingually. The coating layer 36 can also include flavoring to encourage patient compliance. Or, the flavoring can be minimal in the coating, and can be more perceptible in the middle layer 32 to encourage leaving the tablet under the tongue long enough to allow the allergen to dissolve and make substantial contact with the oral mucosa. The patient can be instructed to: “make sure that you leave the tablet under your tongue until you can taste cherries”.

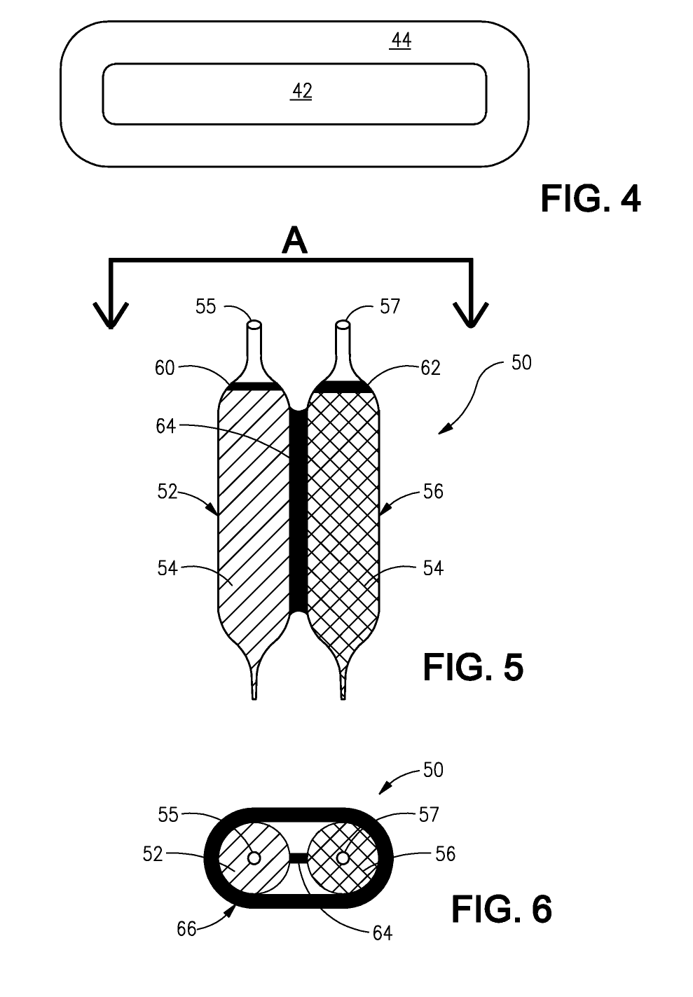

With reference to FIG. 4, a coated tablet 40 has a center 42 and a coating 44. The center 42 contains the allergen, and the coating 44 contains the mast cell stabilizer, such as Cromolyn. In this embodiment, even if the rate of dissolution of the center 42 and the coating 44 are the same, the mast cell stabilizer will still make contact with the oral mucosa before the allergen makes contact with the oral mucosa. Consequently, according to the invention, adverse and/or uncomfortable reactions to the allergen will be reduced and possibly even prevented. The flavoring can be minimal in the coating, and can be more perceptible in the center to encourage leaving the tablet under the tongue long enough to allow the allergen to dissolve and make substantial contact with the oral mucosa. The patient can be instructed to: “make sure that you leave the tablet under your tongue until you can taste cherries”.

Referring to FIG. 5, a pair of squeezable ampoules 52, 56 is shown. The first ampoule 52 can contain a dose of mast cell stabilizer solution, such as Cromolyn sodium solution at a concentration of 4 to 200 mg/ml. The second ampoule 56 can contain a dose of an aqueous extract of an allergen (or combination of allergens). The ampoules 52, 56 can be joined 64 so as to ensure that the dose of the mast cell stabilizer solution is administered in the same locality in the sublingual area as the dose of allergen, via the ampoule openings 55, 57, respectively. To enable the dose of mast cell stabilizer to be delivered BEFORE the dose of the allergen, a pressure seal 60 of the first ampoule 52 is configured to be weaker than the pressure seal 62 of the second ampoule 56, such that when they are both squeezed at the same time, the pressure seal 60 of the first ampoule will break first, thereby releasing the dose of mast cell stabilizer solution before pressure seal 62 of the second ampoule breaks.

Referring to FIG. 6, looking along direction A of FIG. 5, a flexible sleeve 66 surrounds the two squeezable ampoules 52, 56. The flexible sleeve 66 serves to more evenly distribute pressure of squeezing over both ampoules 52, 56, so that steadily applied pressure will pop the different strength pressure seals 60,62 at different times. This enables the mast cell stabilizer solution in the first ampoule 52 to be released BEFORE the allergen solution in the second ampoule 56 is released. This gives the mast cell stabilizer time to work on the oral mucosa where the allergen solution will be released, thereby pre-empting an uncomfortable adverse reaction to the allergen. The allergen solution in the second ampoule 56 can be flavored to encourage complete squeezing and emptying of the pair of ampoules. The flexible sleeve 66 can have a picture or drawing of a colorful fruit (lemon, lime, blueberry, orange, etc.) on each side, indicating the flavor contained therein. For example, if the flavoring is sweet/tart strawberry, there can be a picture of a strawberry on each side of the flexible sleeve 66. While helping the child direct the openings 55,57 of the squeezable ampoules 52, 56 under the tongue, the allergist (or the parent) of a child can say: “Just squeeze the strawberries until you can taste them under your tongue, close your mouth, and then count slowly in your mind up to ten”. This will ensure that the allergen has adequate time to be absorbed by the oral mucosa before swallowing.

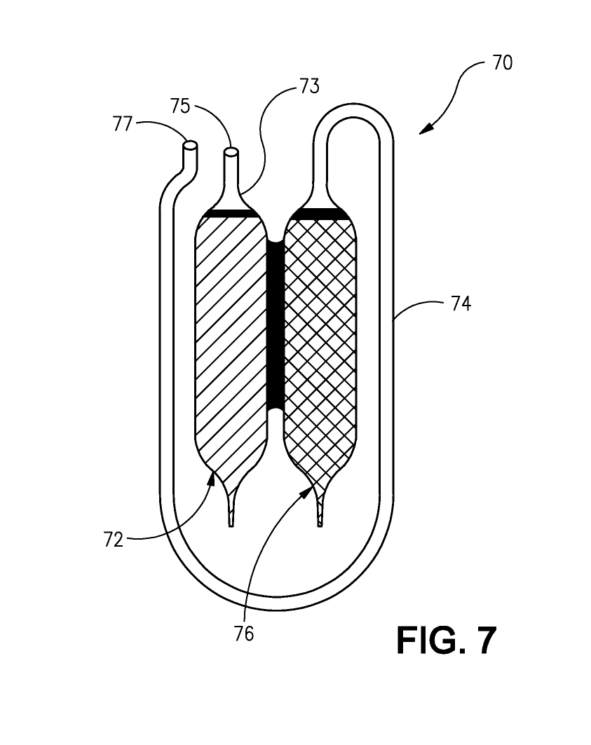

With reference to FIG. 7, a pair of squeezable ampoules 72, 76 each have a pressure seal with the same breaking strength, such that they both pop at about the same time under steady pressure applied by thumb and forefinger. However, the mast cell stabilizer solution contained in the first ampoule 72 is released through an opening 75 having a short channel 73, and the allergen solution is released through an opening 77 having a long channel 74. Because it takes the allergen solution more time to travel through the long channel 74 than it takes for the mast cell stabilizer solution to travel through the short channel 73, the mast cell stabilizer solution makes contact and reacts with the oral mucosa BEFORE the allergen solution released via the long channel 74 makes contact and reacts with the oral mucosa. Consequently, adverse and/or uncomfortable reactions to the allergen are reduced and/or prevented.

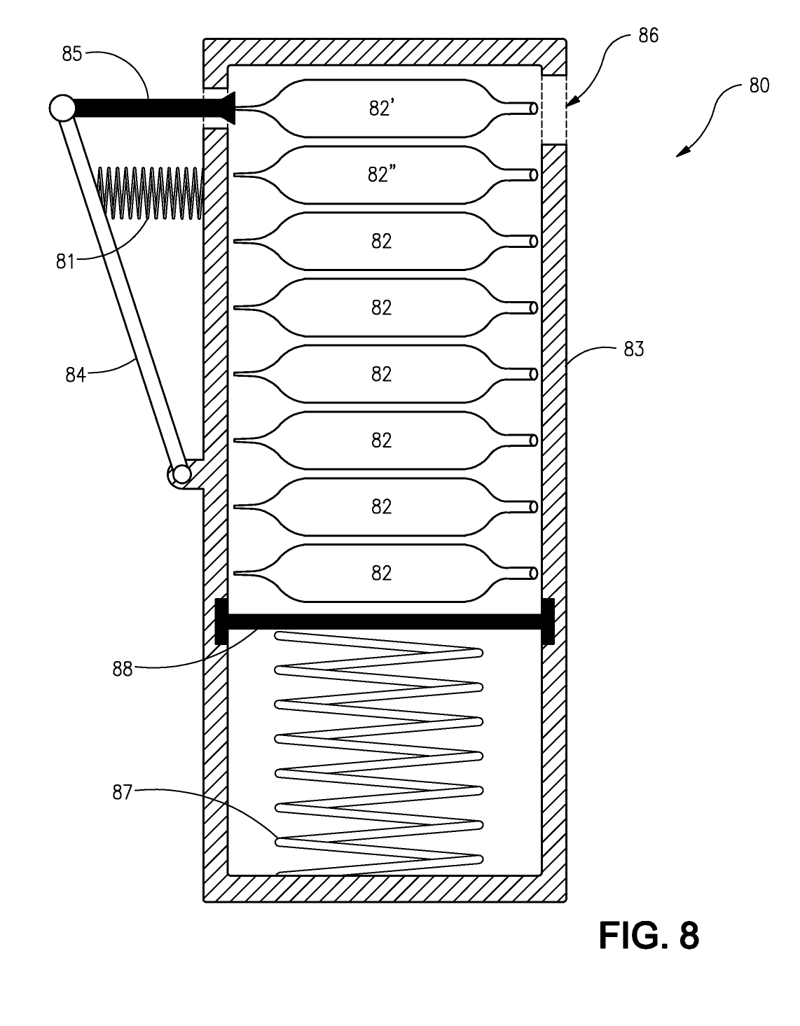

Referring to FIG. 8, a spring-loaded plastic magazine 80 is shown for sequentially dispensing joined pairs of squeezable ampoules 82, so as to provide a sequence of monotonically increasing doses of allergen and a constant dose of mast cell stabilizer. To dispense a top ampoule pair 82′, a user of the magazine 80 holds the outer shell 83 such that the user's thumb can press in a controlled manner upon the plunger lever 84. Pressing on the spring-loaded 81 plunger-lever 84 pushes a plunger 85 against the top ampoule pair 82′ such that the top ampoule pair 82′ is moved out through the opening 86, thereby dispensing the ampoule pair 82′ for use by the user. This allows another ampoule 82″ to move up to the top position (ready to be urged out of the opening 86) under pressure of the spring 87 that continually pushes up upon the pusher plate 88, which serves to lift the entire column of pairs of ampoules 82. Because the ampoules 82 are inserted by the manufacturer in the correct order to provide a monotonically increasing dose of allergen, while also providing a consistent dose of mast cell stabilizer, repeatedly pressing the lever 84 will dispense the ampoule pairs 82 in the correct therapeutic order.

Regarding mechanisms for dispensing an ordered sequence of sublingual tablets or pairs of sublingual tablets, many mechanisms have been taught over the years for dispensing tablets conveniently in sequence. Pill carriers for birth control pills exist for ensuring that pills are taken in the proper order to ensure proper timing of hormones. Spring loaded magazines exist for dispensing candy sequentially, even though the order that the candy is dispensed is of no consequence. According to the invention, however, one can use the same or similar devices to dispense an ordered sequence of fast-dissolving sublingual tablets. Such tablets can be shaped (like a miniature brick, for example) to accommodate existing mechanisms, or such mechanisms can be easily modified to accommodate the rounded or discoid shape of typical sublingual tablets. See for example, U.S. Pat. Nos. 2,620,061; 3,410,455; 3,845,882; 3,942,683; and 4,966,305.

Other modifications and implementations will occur to those skilled in the art without departing from the spirit and the scope of the invention as claimed. Accordingly, the above description is not intended to limit the invention, except as indicated in the following claims.

What is claimed is:

1. A preparation for sublingual immunotherapy, the preparation comprising:
a mast cell stabilizer; and
at least one allergen suitable for sublingual immunotherapy.

2. The preparation of claim 1, wherein the allergen or combination of allergens includes at least one of:
grass pollen, tree pollen, ragweed pollen, weed pollen, dust mite, cat allergen, dog allergen, horse allergen, cow allergen, mice allergen, bird allergen, rabbit allergen, mold spores, hymenoptera venom, cockroach allergen, milk allergen, egg allergen, peanut allergen, nut allergen, seed allergen, wheat allergen, soy allergen, and latex.

3. The preparation of claim 1, wherein the mast cell stabilizer is Cromolyn sodium.

4. The preparation of claim 1, further including a diluent.

5. The preparation of claim 4, wherein the diluent is selected from:
sterile water, glycerin.

6. The preparation of claim 1, further comprising at least one of:
an antihistamine, a leukotriene inhibitor.

7. The preparation of claim 6, wherein the antihistamine is selected from:
diphenhydramine, cetririzine, azelestine, ketotifen, loratidine, olopatatidine, fexofenadine.

8. The preparation of claim 6, wherein the leukotriene inhibitor is montelukast.

9. The preparation of claim 1, wherein the at least one allergen is diluted in a diluent containing a concentration of Cromolyn at 4-200 mg/ml.

10. A multi-layer tablet preparation for sublingual immunotherapy, the preparation comprising:
a first tablet layer including a mast cell stabilizer; and
a second tablet layer including an allergen suitable for sublingual immunotherapy.

11. The tablet preparation of claim 10, wherein the first tablet layer is fast-dissolving such that the first tablet layer dissolves faster than the second tablet layer so that the mast cell stabilizer is rapidly delivered to the sublingual mucosa before most of the allergen makes contact therewith.

12. A multi-layer tablet preparation for sublingual immunotherapy, the preparation comprising:
a first tablet layer including a mast cell stabilizer;
a second tablet layer including an allergen suitable for sublingual immunotherapy; and
a third tablet layer including a mast cell stabilizer.

13. The multi-layer tablet preparation of claim 12, further including:
a dissolvable coating fully enclosing the first, second, and third layers, and wherein the second tablet layer is of a smaller diameter than the first tablet layer and the third tablet layer, so as to ensure that the second layer begins dissolving after the first layer and third layer have started to dissolve.

14. The multi-layer tablet preparation of claim 12, wherein the first layer joins with the third layer so as to enclose the second layer.

15. A tablet preparation for sublingual immunotherapy, the preparation comprising:
a tablet center including an allergen suitable for sublingual immunotherapy; and
a tablet coating, fully enclosing the tablet center, the tablet coating including at least a mast cell stabilizer.

16. An apparatus for administration of sublingual immunotherapy, the apparatus comprising:
a first squeezable ampoule of mast cell stabilizer solution; and
a second squeezable ampoule of an allergen solution.

17. The apparatus of claim 16, further comprising:
a flexible sleeve surrounding the first squeezable ampoule and the second squeezable ampoule, so that when the flexible sleeve is squeezed, the first and second squeezable ampoules are squeezed at substantially the same time, and to substantially the same extent.

18. The apparatus of claim 16, wherein each ampoule is sealed with a pressure-break seal.

19. The apparatus of claim 18, wherein pressure required to break the seal of the first squeezable ampoule containing the mast cell stabilizer is less than the pressure required to break the seal of the squeezable ampoule containing the allergen, such that simultaneously squeezing both ampoules will first break the seal of the first squeezable ampoule containing the mast cell stabilizer, thereby first administering the mast cell stabilizer, and then will break the seal of the second squeezable ampoule containing the allergen solution.

20. The apparatus of claim 18, wherein the thickness of the seal of the first squeezable ampoule is less than the thickness of the second squeezable ampoule, such that the two seals break under different pressure, and therefore at different times.

21. An apparatus for sublingual immunotherapy, the apparatus comprising:
a first ampoule of a solution of mast cell stabilizer;
a second ampoule of a solution of an allergen; and
a mixing nozzle in fluid communication with a pressure seal of the first ampoule and a pressure seal of the second ampoule, such that upon squeezing the first and second ampoules with a pressure sufficient to break both pressure seals, the solution of mast cell stabilizer and the solution of the allergen are ejected into the mixing nozzle, thereby mixing the mast cell stabilizer with the allergen prior to flowing out of the mixing nozzle onto the sublingual mucosa.

22. A method for administration of sublingual immunotherapy, the method comprising:
applying sublingually a sequence of allergen doses of monotonically increasing amount; and
applying sublingually, for each allergen dose of the sequence, a constant dose of mast cell stabilizer.

23. The method of claim 22, wherein the constant dose of mast cell stabilizer is applied sublingually just prior to each dose of the sequence of allergen doses.

24. The method of claim 22, wherein the constant dose of mast cell stabilizer is applied sublingually with each dose of the sequence of allergen doses.

25. A method for performing a dilution sequence of an allergen to produce a sequence of allergen doses of increasing concentration, each allergen dose including a mast cell stabilizer dose at a constant concentration, the method comprising:
obtaining a stock solution of an allergen at a first concentration mixed with a mast cell stabilizer at a selected concentration;
obtaining a first pre-measured vial containing a diluent mixed with the mast cell stabilizer at the selected concentration;
diluting a portion of the stock solution with the contents of the first pre-measured vial to produce a first-dilution solution of the allergen mixed with the mast cell stabilizer at the selected concentration;
obtaining a second pre-measured vial containing the diluent mixed with the mast cell stabilizer at the selected concentration; and
diluting a portion of the first-dilution solution with the contents of the second pre-measured vial to produce a second-dilution of the allergen mixed with the mast cell stabilizer at the selected concentration.

26. The method of claim 25, wherein the mast cell stabilizer is Cromolyn, and the selected concentration is 4-200 mg/ml.

27. The method of claim 25, wherein the diluent is at least one of:
glycerin and water.

28. A stock solution of an allergen at a stock concentration mixed with Cromolyn at a concentration of 4-200 mg/ml.

29. A pre-filled vial of sterile water or glycerine having Cromolyn dissolved therein at a concentration of 4-200 mg/ml for use as a diluent of a stock solution also having the Cromolyn dissolved therein at a concentration of 4-200 mg/ml, the pre-filled vial being usable in a dilution sequence for sublingual immunotherapy.

30. The prefilled vial of claim 29, prefilled to a volume selected from: 9 ml, 4.5 ml, 1.8 ml.

31. A dosing system for providing sublingual immunotherapy via sublingual tablets, the dosing system comprising:
a therapeutic sequence of sublingual tablets, the therapeutic sequence of sublingual tablets having a monotonically increasing dose of allergen, and a same dose of mast cell stabilizer in each tablet of the therapeutic sequence; and
a plurality of maintenance dose sublingual tablets, each maintenance dose sublingual tablet having a same maintenance dose of allergen, and the same dose of mast cell stabilizer as in each sublingual tablet of the therapeutic sequence.

32. The dosing system of claim 31, wherein each sublingual tablet includes a dose of Cromolyn of 4-200 mg.

33. The dosing system of claim 31, wherein each sublingual tablet dissolves within 5 to 120 seconds.

34. The dosing system of claim 31, wherein each sublingual tablet can be taken soon after taking a preventative tablet of Cromolyn of 4-200 mg to prevent an uncomfortable oral allergic reaction to the allergen.

35. The dosing system of claim 31, wherein each sublingual tablet can be followed promptly by a therapeutic tablet of Cromolyn of 4-200 mg to treat an uncomfortable oral allergic reaction to the allergen.

36. A dosing system for providing sublingual immunotherapy via sublingual ampoules, the dosing system comprising:
a therapeutic sequence of sublingual ampoules, the therapeutic sequence of sublingual ampoules having a monotonically increasing dose of allergen, and a same dose of mast cell stabilizer in each ampoule of the therapeutic sequence; and
a plurality of maintenance dose sublingual ampoules, each maintenance dose sublingual ampoule having a same maintenance dose of allergen, and the same dose of mast cell stabilizer as in each sublingual ampoule of the therapeutic sequence.

37. The dosing system of claim 36, wherein the therapeutic sequence of sublingual ampoules is contained in a spring-loaded magazine so that the therapeutic sequence of sublingual ampoules can be dispense in sequence.

38. Apparatus for sequentially dispensing a therapeutic sequence of sublingual ampoules, the apparatus comprising:
a spring-loaded magazine for dispensing joined pairs of squeezable ampoules in a sequence having monotonically increasing dose of allergen and constant dose of mast cell stabilizer; and
a stack of joined pairs of squeezable ampoules in a sequence having monotonically increasing dose of allergen and constant dose of mast cell stabilizer.

39. A method for sublingual immunotherapy, the method comprising:
taking a sequence of allergen tablets of monotonically increasing dose; and
taking a constant sublingual dose of cromolyn 4-200 mg along with each allergen tablet of the sequence.

40. The method of claim 39, wherein taking the constant sublingual dose of cromolyn 4-200 mg is taken in the form of: sublingual tablet; liquid; spray; and drops.
